(19) United States
(12) Patent Application Publication
(10) Pub. No.: US 2024/0218345 A1
(43) Pub. Date: Jul. 4, 2024

(54) DECOLORIZING COMPOSITION INCLUDING DICYANDIAMIDE AND HYDROGEN PEROXIDE AND DECOLORIZING METHOD USING THE SAME

(71) Applicant: CJ CHEILJEDANG CORPORATION, Seoul (KR)

(72) Inventors: Jung Won PARK, Seoul (KR); Hyo Hyoung LEE, Seoul (KR); Sungkyun LEE, Seoul (KR); Young Kyung KIM, Seoul (KR); Taek Beom KIM, Seoul (KR); Chang Jun JI, Seoul (KR); Yu Shin KIM, Seoul (KR); Minhoe KIM, Seoul (KR)

(73) Assignee: CJ CHEILJEDANG CORPORATION, Seoul (KR)

(21) Appl. No.: 18/402,269

(22) Filed: Jan. 2, 2024

(30) Foreign Application Priority Data
Jan. 3, 2023 (KR) 10-2023-0000841

(51) Int. Cl. C12N 9/88; C12N 1/00; C12N 1/20; C12N 9/96; C12R 1/07

(52) U.S. Cl. CPC C12N 9/88; C12N 1/005; C12N 1/20; C12N 9/96; C12R 2001/07; C12Y 402/02002

(57) ABSTRACT

Provided are a method of decolorizing a composition including pectate lyase, and a decolorizing composition for the composition including pectate lyase.

### BACKGROUND OF THE INVENTION

### 1. Field of the Invention

The present disclosure relates to a method of decolorizing a composition including pectate lyase, and a composition for decolorizing the composition including pectate lyase.

### 2. Description of the Related Art

Pectin is a group of complex heteropolymers, present in the middle lamella of the primary plant-cell wall, and act as intercellular cement. They also have an important function in the water-regulation of plants due to their colloidal nature. The middle lamellas which are situated between the cell walls are mainly built up from protopectin (an insoluble form of pectin).

Pectin is composed of two different definite regions: “smooth” and “hairy” regions. The “smooth” region consists of a backbone of α-1,4-linked D-galacturonic acid, forming a polygalacturonic acid with some of carboxyl groups esterified with methanol (Rouse A. Pectin: distribution, significance. In: Nagy S, Shaw P, Veldhuis Meds. Citrus Science and Technology, Vol I. Westport CT: AVI publishing Inc. 1977). In the “hairy” region, the galacturonic acid backbone is broken down by α-1,2-linked L-rhamnose unit.

Pectin/pectate lyases depolymerizes pectin in the smooth region, which cleaves glycosidic bonds via ß-elimination to yield oligomers that are 4,5-unsaturated non-reducing ends (Lombard V, Bernard T, Rancurel C, Brumer H, Coutinho P M, Henrissat B. A hierarchical classification of polysaccharide lyases for glycogenomics. Biochem J. 2010; 432(3):437-444). Pectate lyase is used in a variety of industrial fields, such as paper manufacture, textile manufacture, etc., and in this regard, WO 98/45393 discloses detergent compositions containing protopectinase with remarkable detergency against muddy soiling.

Meanwhile, in general, a fermentation process of using microorganisms is mainly used in the production of enzymes. However, there is a problem in that quality of the product deteriorates due to dyes generated as fermentation by-products, and there is a problem in that deactivation of the enzyme, which loses its activity as an enzyme, occurs during a decolorizing process to remove the dyes.

In this regard, U.S. Pat. No. 5,869,440A discloses a decolorizing agent for removing mold, the decolorizing agent including hydrogen peroxide and dicyandiamide. However, the prior literature does not mention a method of preventing deactivation of the enzyme, which occurs during the decolorizing process in the enzyme production.

Therefore, research is still needed to effectively improve a color value while preventing deactivation of enzymes, which occurs during the decolorizing process when producing the enzymes.

### SUMMARY OF THE INVENTION

An object of the present disclosure is to provide a method of decolorizing a composition including pectate lyase, the method including the steps of preparing the composition including pectate lyase by culturing a microorganisms of the genus Bacillus in a medium; and decolorizing the composition including pectate lyase by adding dicyandiamide and hydrogen peroxide thereto.

Another object of the present disclosure is to provide a composition for decolorizing a composition including pectate lyase, the composition including dicyandiamide and hydrogen peroxide.

### BRIEF DESCRIPTION OF THE DRAWINGS

FIG. 1 shows results of examining decolorizing effects of dicyandiamide and hydrogen peroxide according to concentrations.

### DETAILED DESCRIPTION OF THE PREFERRED EMBODIMENTS

The present disclosure will be described in detail as follows. Meanwhile, each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description described below.

Further, those skilled in the art will recognize, or be able to ascertain using no more than routine experimentation, many equivalents to the specific embodiments of the disclosure described herein. Further, these equivalents should be interpreted to fall within the present disclosure.

As used in the specification and appended claims, the singular forms (“a”, “an”, and “the”) include plural referents unless the context clearly states otherwise. Unless the context indicates otherwise, singular terms shall include the plural and plural terms shall include the singular. As used in the specification and appended claims, unless stated otherwise, the use of “or” may be used to include “and/or”.

As used herein, the term “about” may be presented before a particular numerical value. The term “about” used herein includes not only the exact number recited after the term, but also a range that is near or close to that number. Considering the context in which the number is presented, it can be determined whether any number is close to or near the particular number presented. In one example, the term “about” may refer to a range from −10% to +10% of a numerical value. In another example, the term “about” may refer to a range from −5% to +5% of a given numerical value, but is not limited thereto.

As used herein, the descriptions such as the terms “first, second, third . . . ” “i), ii), iii) . . . ” or “(a), (b), (c), (d) . . . ” are used to distinguish similar constitutions, and these terms do not mean that the constitutions are performed continually or sequentially. For example, when the terms are used in reference to steps of a method, use, or assay, there may be no time interval between these steps, or they may be performed concurrently, or may be performed several seconds, several minutes, several hours, several days, or several months apart.

As used herein, the term “comprising/including” means the presence of features, steps or components recited after the term, and does not exclude the presence or addition of one or more features, steps or components. The components or features recited after the term “comprising/including” herein may be essential or mandatory. However, in some embodiments, the term may further include any other or non-essential components or features.

An aspect of the present disclosure provides a method of decolorizing a composition including pectate lyase, the method including the steps of preparing the composition including pectate lyase by culturing a microorganisms of the genus Bacillus in a medium; and decolorizing the composition including pectate lyase by adding dicyandiamide and hydrogen peroxide thereto.

In general, a fermentation process of using microorganisms is mainly used in the production of enzymes. However, there is a problem in that quality of the product deteriorates due to dyes generated as fermentation by-products, and there is a problem in that deactivation of the enzyme, which loses its activity as an enzyme, occurs during a decolorizing process to remove the dyes.

The method of decolorizing the composition including pectate lyase of the present disclosure has excellent effects in that dicyandiamide and hydrogen peroxide are employed to effectively remove dyes generated as fermentation by-products during the decolorizing process in the production of enzymes, while solving the problem of deactivation of the enzyme during the decolorizing process, thereby increasing the process yield and economic efficiency.

In the present disclosure, one standard unit (U) of enzyme activity may be defined as the amount of the enzyme required to produce 1 μmol of unsaturated polygalacturonic acid per minute by cleaving polygalacturonic acid, but is not limited thereto. Unsaturated galacturonic acid may be used interchangeably with D-galacturonic acid. For example, in the present disclosure, 1 unit (U) of enzyme activity is defined as the amount of the enzyme required to release 1 μmol of unsaturated polygalacturonic acid per minute from a polygalacturonic acid solution with a concentration of 6 mg/mL at 50° C. and pH 7.73, but is not limited to thereto.

As used herein, the term “pectate lyase” depolymerizes pectin in the smooth region, which cleaves glycosidic bonds via β-elimination to yield oligomers that are 4,5-unsaturated non-reducing ends (Lombard V, Bernard T, Rancurel C, Brumer H, Coutinho P M, Henrissat B. A hierarchical classification of polysaccharide lyases for glycogenomics. Biochem J. 2010; 432(3):437-444). Pectate lyase is used in a variety of industrial fields, such as paper manufacture, textile manufacture, etc.

As used herein, the term “dicyandiamide” may be used interchangeably with “dicyandiamide polymer”.

The dicyandiamide and hydrogen peroxide used in the present disclosure are not particularly limited, and dicyandiamide and hydrogen peroxide commonly used in the relevant technical field or similar fields may be used.

Specifically, the method of decolorizing the composition including pectate lyase of the present disclosure will be described in detail.

First, the composition including pectate lyase is prepared by culturing a microorganism of the genus Bacillus in a medium.

In one specific embodiment, the composition including pectate lyase may include a microorganism of the genus Bacillus, a culture of the microorganism, a fermentation product of the microorganism, or a combination of two or more thereof.

As used herein, the term “culturing” means growing the strain of the present disclosure under appropriately controlled environmental conditions. The culturing process of the present disclosure may be carried out according to appropriate medium and culture conditions known in the art. This culturing process may be easily adjusted and used by a person skilled in the art, depending on the strain selected. Specifically, the culturing may be carried out in a batch, continuous, and/or fed-batch manner, but is not limited thereto.

As used herein, the term “medium” refers to a mixture of materials which contains nutrient materials required for the culturing of the microorganism as a main ingredient, and it supplies nutrient materials and growth factors, along with water that is essential for survival and growth. Specifically, the medium and other culture conditions used for culturing the strain of the present disclosure may be any common medium used for culturing of microorganisms without any particular limitation. However, the microorganism of the present disclosure may be cultured under aerobic conditions in a common medium containing appropriate carbon sources, nitrogen sources, phosphorus sources, inorganic compounds, amino acids, and/or vitamins, while adjusting temperature, pH, etc.

In the present disclosure, the carbon sources may include carbohydrates, such as glucose, saccharose, lactose, fructose, sucrose, maltose, etc.; sugar alcohols, such as mannitol, sorbitol, etc.; organic acids, such as pyruvic acid, lactic acid, citric acid, etc.; amino acids, such as glutamic acid, methionine, lysine, etc. Further, natural organic nutrients such as starch hydrolysate, molasses, blackstrap molasses, rice bran, cassava, sugar cane molasses, and corn steep liquor, etc. may be used. Specifically, carbohydrates such as glucose and sterilized pretreated molasses (i.e., molasses converted to reducing sugar) may be used, and in addition, various other carbon sources may be used in an appropriate amount without limitation. These carbon sources may be used alone or in a combination of two or more thereof, but are not limited thereto.

The nitrogen sources may include inorganic nitrogen sources, such as ammonia, ammonium sulfate, ammonium chloride, ammonium acetate, ammonium phosphate, ammonium carbonate, ammonium nitrate, etc.; organic nitrogen sources, such as amino acids, e.g., glutamic acid, methionine, glutamine, etc., peptone, NZ-amine, meat extract, yeast extract, malt extract, corn steep liquor, casein hydrolysate, fish or decomposition product thereof, defatted soybean cake or decomposition product thereof, etc. These nitrogen sources may be used alone or in a combination of two or more thereof, but are not limited thereto.

The phosphorus sources may include monopotassium phosphate, dipotassium phosphate, or corresponding sodium-containing salts, etc. The inorganic compounds may include sodium chloride, calcium chloride, iron chloride, magnesium sulfate, iron sulfate, manganese sulfate, calcium carbonate, etc. In addition, amino acids, vitamins, and/or appropriate precursors may be included. These components or precursors may be added to a medium in a batch or continuous manner, but are not limited thereto.

Further, pH of the medium may be adjusted by adding a compound such as ammonium hydroxide, potassium hydroxide, ammonia, phosphoric acid, sulfuric acid, etc. during the culturing of the microorganism in an appropriate manner. In addition, bubble formation may be prevented during the culturing using an antifoaming agent such as fatty acid polyglycol ester. Further, oxygen or an oxygen-containing gas may be injected to the medium in order to maintain aerobic conditions of the medium; or no gas may be injected, or nitrogen, hydrogen, or carbon dioxide gas may be injected in order to maintain anaerobic or microaerobic conditions, but is not limited thereto.

In the culturing of the present disclosure, the culturing temperature may be maintained at 27° C. to 37° C., specifically, at 30° C. to 33° C., and the culturing may be carried out for about 20 hours to 120 hours, but is not limited thereto.

As used herein, the term “culture” means a culture broth, a concentrated culture broth, a dried product of the culture broth, a culture filtrate, a concentrated culture filtrate, or a dried product of the culture filtrate, which is obtained by culturing a specific microorganism in a culture medium. The culture broth means those containing a specific microorganism, and the culture filtrate means substantially not including the specific microorganism (here, it means substantially excluding the specific microorganism to be separated by filtration, etc., but does not mean that the microorganism is completely excluded from the filtrate). The formulation of the culture is not limited, and may be, for example, a liquid, an emulsion, or a solid. Specifically, with respect to the objects of the present disclosure, the culture may include L-alanine.

As used herein, the term “fermentation” refers to a process in which microorganisms break down organic materials using their own enzymes, excluding putrefaction. Fermentation and putrefaction proceed by similar processes, but when useful substances are produced as a result of decomposition, it is called fermentation, and when bad smells or harmful substances are produced, it is called putrefaction.

In the present disclosure, the method of obtaining the fermentation product from the strain is not particularly limited, and the fermentation product may be obtained according to a method commonly used in the art or similar art.

In the present disclosure, the term “fermentation product” includes not only a fermented material itself, but also all types of materials including the fermentation product generated from the strain, such as a culture medium of a strain, in which the strain and the culture coexist, a fermentation product obtained by filtering the strain from the culture medium, a fermentation product obtained by sterilizing the strain from the culture medium and filtering the same, an extract obtained by extracting the fermentation product or the culture medium including the same, a dilution obtained by diluting the fermentation product or the extract thereof, a concentrate, a dry product obtained by drying the fermentation product or the extract thereof, a lysate obtained by collecting and disrupting cells of the strain, etc.

In the method of the present disclosure, the culturing of the microorganism may be carried out using any culturing conditions and culturing method known in the art. This culturing process may be easily adjusted and used by a person skilled in the art, depending on the strain selected.

Subsequently, the composition including pectate lyase is decolorized by adding dicyandiamide and hydrogen peroxide thereto.

In one specific embodiment, the dicyandiamide may be included in an amount of 0.1 part by weight to 5 parts by weight, specifically, 0.1 part by weight to 4.5 parts by weight, and more specifically, 0.5 parts by weight to 4 parts by weight, based on the total 100 parts by weight of the composition, but is not limited thereto.

In one specific embodiment, the hydrogen peroxide may be included in an amount of 0.1 M to 1 M, specifically, 0.5 M to 1 M, but is not limited thereto.

When the dicyandiamide and hydrogen peroxide are included in the above range, the color value may be effectively improved while preventing deactivation of the enzyme which occurs in the decolorizing process during the production of enzyme. In particular, it is the optimal amount to produce the maximum decolorizing effect while minimizing the addition amounts of dicyandiamide and hydrogen peroxide, and is effective in terms of production cost and process design, as compared to processes of using activated carbon, resin towers, etc. which are used in the existing decolorizing process.

In the method of decolorizing the composition including pectate lyase of the present disclosure, the composition including dicyandiamide and hydrogen peroxide may have increased stability of pectate lyase after the decolorizing step, as compared to a composition not including dicyandiamide and hydrogen peroxide.

Specifically, the composition comprising pectate lyase after the decolorizing step may maintain 90% or more of the activity of pectate lyase, as compared to the composition before the decolorizing step.

Still another aspect of the present disclosure provides a composition for decolorizing the composition including pectate lyase, the composition including dicyandiamide and hydrogen peroxide.

The dicyandiamide, hydrogen peroxide, and pectate lyase are as described above.

In one specific embodiment, the pectate lyase may be derived from a microorganism of the genus Bacillus.

In one specific embodiment, the composition including pectate lyase may include the microorganism of the genus Bacillus, a culture of the microorganism, a fermentation product of the microorganism, or a combination of two or more thereof.

In one specific embodiment, the dicyandiamide may be included in an amount of 0.1 part by weight to 5 parts by weight, specifically, 0.1 part by weight to 4.5 parts by weight, and more specifically, 0.5 parts by weight to 4 parts by weight, based on the total 100 parts by weight of the composition, but is not limited thereto.

In one specific embodiment, the hydrogen peroxide may be included in an amount of 0.1 M to 1 M, specifically, 0.5 M to 1 M, but is not limited thereto.

When the dicyandiamide and hydrogen peroxide are included in the above range, the color value may be effectively improved while preventing deactivation of the enzyme which occurs in the decolorizing process during the production of enzyme.

The composition for decolorizing the composition including pectate lyase of the present disclosure may have improved stability of pectate lyase, as compared to a composition not including dicyandiamide and hydrogen peroxide.

Hereinafter, the present disclosure will be described in more detail by way of exemplary embodiments. However, the following exemplary embodiments are only preferred embodiments for illustrating the present disclosure, and thus are not intended to limit the scope of the present disclosure thereto. Meanwhile, technical matters not described in the present specification may be sufficiently understood and easily implemented by those skilled in the technical field of the present disclosure or similar technical fields.

### Comparative Example 1. Evaluation of Decolorizing Effect and Enzyme Deactivation Prevention Effect of Dicyandiamide

Dicyandiamide (Dongguan Sanmei Chemical Technology Co., Ltd) was added at a concentration of 0% to 4% to a fermentation concentrate (enzyme activity of 480,000 Unit/mL) of Bacillus platform (Bacillus subtilis) pectate lyase.

In the production of the pectate lyase enzyme by fed-batch culture, dissolved oxygen (DO) was used as an indicator, and a feeding medium was supplied at the time when DO rose. At this time, the ingredients of the main fermentation medium were composed of maltose, glucose, yeast extract, corn steep liquor, calcium chloride, monobasic sodium phosphate, ammonium chloride, and magnesium sulfate. The feeding medium was composed of maltose, yeast extract, and ammonium chloride. Meanwhile, fermentation conditions were maintained at a culture temperature of 37° C. while maintaining pH at 6.7 to 7.2 using ammonia water. The stirring speed was adjusted to 600 rpm to 1,000 rpm to avoid DO limitations. The final culture medium was subjected to filter press (F/P) to separate bacterial cells, which were then concentrated by ultrafiltration (U/F).

Subsequently, after the reaction was allowed at room temperature for 30 minutes, a color value (T-C %, 240 nm) and enzyme activity were measured. In detail, the color value was measured using a spectrophotometer (Shimadzu Instruments Co., Ltd., UV-19001), and 1 unit (U) of enzyme activity of pectate lyase was determined as the amount of the enzyme required to release 1 μmol of unsaturated polygalacturonic acid per minute from a polygalacturonic acid solution with a concentration of 6 mg/mL at 50° C. and pH 7.73. The activity residual rate is expressed as a percentile of the enzyme activity value measured after the decolorizing process relative to the initial enzyme activity value.

The results are shown in Table 1 below.

TABLE 1

| | | | T-C % (Abs | Enzyme activity |
|---|---|---|---|---|
| | | Dicyandiamide | at 420 | residual rate |
| Type | Sample | % | nm) | % |
| Fermentation | 1 | 0.0 | 0.27 | 100 |
| broth | 2 | 0.5 | 0.49 | 100 |
| | 3 | 1.0 | 0.74 | 100 |
| | 4 | 2.0 | 1.35 | 99 |
| | 5 | 3.0 | 1.70 | 95 |
| | 6 | 4.0 | 1.94 | 99 |

As shown in Table 1, it was confirmed that when dicyandiamide was used, the enzyme was not deactivated up to a concentration of 4%, but T-C % was 5 or less, indicating the weak decolorizing effect.

### Comparative Example 2. Evaluation of Decolorizing Effect and Enzyme Deactivation Prevention Effect of Hydrogen Peroxide

Hydrogen peroxide (30% aqueous solution, H₂O₂, Sinopharm Chemical Reagent Co., Ltd) was added at a concentration of 0 M to 4 M to the fermentation concentrate of Bacillus platform pectate lyase of Comparative Example 1.

Subsequently, after the reaction was allowed at room temperature, a color value (T-C %, 240 nm) and enzyme activity were measured every 24 hours. The results are shown in Table 2 below.

TABLE 2

| | | 30% | T-C % | | | Enzyme activity | | |
|---|---|---|---|---|---|---|---|---|
| | Sam- | H₂O₂ | (Abs at 420 nm) | | | residual rate (%) | | |
| Type | ple | M | 24 hr | 48 hr | 72 hr | 24 hr | 48 hr | 72 hr |
| Fermen- | 1 | 0.0 | 0.30 | 0.31 | 0.26 | 97 | 89 | 77 |
| tation | 2 | 0.5 | 0.32 | 0.30 | 0.24 | 100 | 97 | 98 |
| broth | 3 | 1.0 | 1.23 | 1.81 | 2.01 | 100 | 100 | 99 |
| | 4 | 2.0 | 11.92 | 20.82 | — | 12 | 1 | Not |
| | | | | | | | | measu- |
| | | | | | | | | rable |
| | 5 | 3.0 | 8.73 | 11.23 | — | 2 | 1 | Not |
| | | | | | | | | measu- |
| | | | | | | | | rable |

As shown in Table 2, it was confirmed that the condition under which the enzyme was not deactivated by H₂O₂ was the use of 1.0% or less, but T-C % was 5 or less, indicating the weak decolorizing effect.

### Example 1. Evaluation of Decolorizing Effect and Enzyme Deactivation Prevention Effect of Dicyandiamide and Hydrogen Peroxide-1

The concentration of dicyandiamide was fixed at 2% in the fermentation concentrate of Bacillus platform pectate lyase of Comparative Example 1 and H₂O₂ was added thereto at a concentration of 0.8 M to 1.8 M.

Subsequently, after the reaction was allowed at room temperature, a color value (T-C %, 240 nm) and enzyme activity were measured every 24 hours. The results are shown in Table 3 below.

TABLE 3

| | | | 30% | | | | Enzyme | | |
|---|---|---|---|---|---|---|---|---|---|
| | | Dicyandiamide | H₂O₂ | T-C% (Abs at 420 nm) | | | activity residual rate (%) | | |
| Type | Sample | % | M | 24 hr | 48 hr | 72 hr | 24 hr | 48 hr | 72 hr |
| Fermentation | 1 | 0.0 | 0.0 | 0.21 | 0.19 | 0.21 | 100 | 99 | 100 |
| broth | 2 | 2.0 | 0.8 | 3.70 | 4.15 | 4.00 | 100 | 100 | 100 |
| | 3 | 2.0 | 1.0 | 10.22 | 13.93 | 16.37 | 100 | 100 | 97 |
| | 4 | 2.0 | 1.2 | 15.95 | 22.19 | 27.92 | 100 | 77 | 59 |
| | 5 | 2.0 | 1.4 | 20.54 | 31.06 | 34.93 | 94 | 45 | 12 |
| | 6 | 2.0 | 1.6 | 23.87 | 38.19 | 44.53 | 80 | 26 | 6 |
| | 7 | 2.0 | 1.8 | 34.18 | 47.18 | 52.24 | 16 | 10 | 1 |

As shown in Table 3, it was confirmed that the enzyme was not deactivated under conditions of 0.8 M to 1.0 M of H₂O₂ and 2% dicyandiamide, and it was confirmed that the color value was improved by 10 times or more, relative to the fermentation broth (Sample 1 in Table 3) before applying the decolorizing process.

It was also confirmed that the color value was improved by about 8 times or more under conditions of 1.0 M of H₂O₂ and 2% dicyandiamide, relative to the condition of Comparative Example 2 where H₂O₂ was used alone.

### Example 2. Evaluation of Decolorizing Effect and Enzyme Deactivation Prevention Effect of Dicyandiamide and Hydrogen Peroxide-2

The concentration of H₂O₂ was fixed at 1.0 M in the fermentation concentrate of Bacillus platform pectate lyase of Comparative Example 1 and dicyandiamide was added thereto at a concentration of 0% to 4%.

Subsequently, after the reaction was allowed at room temperature, a color value (T-C %, 240 nm) and enzyme activity were measured every 24 hours. The results are shown in Table 4 below.

TABLE 4

| | | | 30% | | | | Enzyme | | |
|---|---|---|---|---|---|---|---|---|---|
| | | Dicyandiamide | H₂O₂ | T-C % (Abs at 420 nm) | | | activity residual rate (%) | | |
| Type | Sample | % | M | 24 hr | 48 hr | 72 hr | 24 hr | 48 hr | 72 hr |
| Fermentation | 1 | 0.0 | 1.0 | 0.75 | 1.02 | 1.13 | 100 | 100 | 100 |
| broth | 2 | 0.5 | 1.0 | 4.28 | 5.65 | 6.00 | 100 | 100 | 100 |
| | 3 | 1.0 | 1.0 | 5.65 | 6.63 | 7.70 | 100 | 100 | 99 |
| | 4 | 1.5 | 1.0 | 7.48 | 8.80 | 9.54 | 100 | 100 | 99 |
| | 5 | 2.0 | 1.0 | 8.83 | 9.34 | 10.78 | 100 | 100 | 99 |
| | 6 | 2.5 | 1.0 | 9.71 | 11.14 | 11.39 | 100 | 100 | 99 |
| | 7 | 3.0 | 1.0 | 9.70 | 10.69 | 11.21 | 100 | 100 | 99 |
| | 8 | 4.0 | 1.0 | 12.32 | 13.76 | 12.40 | 100 | 100 | 96 |

As shown in Table 4, it was confirmed that the enzyme was not deactivated under all conditions of 1.0 M of H₂O₂ and 0% to 4% dicyandiamide, and the decolorizing effect was obtained.

### Example 3. Evaluation of Decolorizing Effect and Enzyme Deactivation Prevention Effect of Dicyandiamide and Hydrogen Peroxide-3

Next, it was intended to examine whether the decolorizing effect and the enzyme deactivation prevention effect could be obtained even though dicyandiamide and hydrogen peroxide were added at low concentrations.

H₂O₂ was added at 0.5 M or 0.8 M to the fermentation concentrate of Bacillus platform pectate lyase of Comparative Example 1 and dicyandiamide was added thereto at a concentration of 0% to 1.5%.

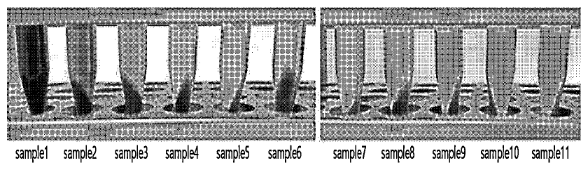

Subsequently, after the reaction was allowed at room temperature, a color value (T-C %, 240 nm) and enzyme activity were measured every 24 hours. The results are shown in Table 5 below and FIG. 1.

TABLE 5

| | | | | | | Enzyme | |
|---|---|---|---|---|---|---|---|
| | | | | | | activity | |
| | | | 30% | T-C % (Abs at | | residual rate | |
| | Sam- | Dicyandiamide | H₂O₂ | 420 nm) | | (%) | |
| Type | ple | % | M | 48 hr | 72 hr | 48 hr | 72 hr |
| Fermen- | 1 | 0.00 | 0.0 | 0.23 | 0.25 | 97 | 99 |
| tation | 2 | 0.50 | 0.5 | 0.81 | 0.68 | 100 | 99 |
| broth | 3 | 0.75 | 0.5 | 1.82 | 1.67 | 100 | 99 |
| | 4 | 1.00 | 0.5 | 2.51 | 2.54 | 100 | 99 |
| | 5 | 1.25 | 0.5 | 3.39 | 3.73 | 100 | 99 |
| | 6 | 1.50 | 0.5 | 3.52 | 3.20 | 100 | 99 |
| | 7 | 0.50 | 0.8 | 5.88 | 7.75 | 100 | 75 |
| | 8 | 0.75 | 0.8 | 8.84 | 11.01 | 100 | 91 |
| | 9 | 1.00 | 0.8 | 10.60 | 11.61 | 100 | 87 |
| | 10 | 1.25 | 0.8 | 12.91 | 11.15 | 100 | 91 |
| | 11 | 1.50 | 0.8 | 13.58 | 12.41 | 100 | 90 |

As shown in Table 5 and FIG. 1, it was confirmed that even when dicyandiamide and hydrogen peroxide were added at low concentrations, the enzyme was not deactivated and the excellent decolorizing effect was obtained.

### Example 4. Evaluation of Decolorizing Effect and Enzyme Deactivation Prevention Effect of Dicyandiamide and Hydrogen Peroxide in Fermentation Concentrates of Other Enzymes than Pectate Lyase

It was intended to examine the decolorizing effect and the enzyme deactivation prevention effect of dicyandiamide and hydrogen peroxide in fermentation concentrates of other enzymes than pectate lyase.

The final fermentation broths of Bacillus platform (Bacillus subtilis) enzymes, amylase and alkaline protease enzymes were subjected to filter press (F/P) to separate bacterial cells, which were then concentrated by ultrafiltration (U/F) to reach a concentration of 480,000 Unit/mL. H₂O₂ was added at a concentration of 0.5 M or 0.8 M to the enzyme fermentation concentrates, and dicyandiamide was added at a concentration of 0% to 1.5%.

In the production of the amylase enzyme by fed-batch culture, dissolved oxygen (DO) was used as an indicator, and a feeding medium was supplied at the time when DO rose. At this time, the ingredients of the main fermentation medium were composed of glucose, yeast extract, corn steep liquor, calcium chloride, monobasic sodium phosphate, ammonium chloride, manganese sulfate, iron sulfate, and zinc sulfate. The feeding medium was composed of glucose, yeast extract, and ammonium chloride. Meanwhile, fermentation conditions were maintained at a culture temperature of 37° C. while maintaining pH at 6.7 to 7.2 using ammonia water. The stirring speed was adjusted to 600 rpm to 1,000 rpm to avoid DO limitations.

In the production of the alkaline protease enzyme by fed-batch culture, dissolved oxygen (DO) was also used as an indicator, and a feeding medium was supplied at the time when DO rose. At this time, the ingredients of the main fermentation medium were composed of glucose, dextrin, yeast extract, corn steep liquor, soy peptone, calcium chloride, monobasic sodium phosphate, ammonium chloride, manganese sulfate, iron sulfate, zinc sulfate, and magnesium sulfate. The feeding medium was composed of maltose, soy peptone, yeast extract, and ammonium chloride. The fermentation conditions were maintained at a culture temperature of 37° C. while maintaining pH at 6.7 to 7.2 using ammonia water. The stirring speed was adjusted to 600 rpm to 1,000 rpm to avoid DO limitations.

Subsequently, the reaction was allowed at room temperature, and after 72 hours, a color value (T-C %, 240 nm) and enzyme activity were measured every 24 hours. The results are shown in Table 6 below.

TABLE 6

| | | | | | | | Enzyme activity residual rate (%) | | |
|---|---|---|---|---|---|---|---|---|---|
| | | | 30% | T-C % (Abs at 420 nm) | | | | | Alkaline |
| | Sam- | Dicyandiamide | H₂O₂ | Pectate | | Alkaline | Pectate | Amylase | protease |
| Type | ple | % | M | lyase | Amylase | protease | lyase | (%) | (%) |
| Fermen- | 1 | 0.0 | 0.0 | 0.24 | 0.18 | 0.32 | 100 | 99 | 100 |
| tation | 2 | 0.5 | 0.5 | 0.73 | 0.65 | 0.75 | 100 | 83 | 79 |
| broth | 3 | 1.0 | 0.5 | 2.86 | 2.82 | 2.35 | 99 | 81 | 81 |
| | 4 | 1.5 | 0.5 | 3.65 | 3.42 | 3.15 | 99 | 82 | 80 |
| | 5 | 0.5 | 0.8 | 8.02 | 6.95 | 8.12 | 92 | 76 | 78 |
| | 6 | 1.0 | 0.8 | 12.02 | 11.29 | 11.89 | 91 | 78 | 69 |
| | 7 | 1.5 | 0.8 | 13.62 | 12.92 | 11.90 | 93 | 71 | 73 |

As shown in Table 6, it was confirmed that even though dicyandiamide and hydrogen peroxide were added to other enzymes than pectate lyase, only the decolorizing effect was exhibited, and the enzyme activity was deactivated. Accordingly, it was confirmed that the decolorizing composition for the composition including pectate lyase of the present disclosure exhibits the selective effect on pectate lyase.

### Example 5. Evaluation of Enzyme (Pectate Lyase) Performance after Decolorizing Process

It was intended to verify the performance of the enzyme (pectate lyase) after the decolorizing process. Specifically, application to paper manufacture was evaluated before/after decolorizing.

In detail, 30 g of whole dry pulp was first taken and mixed with appropriate amounts of enzyme solution and water to obtain a 10% pulp concentration. Next, it was reacted in a water bath at 40° C. for 60 minutes, and after completion of the reaction, sterilization was carried out for 20 minutes through water bathing at 96° C. After pulping, decomposition, and dilution processes, the degree of dissociation of the sample was measured, and physical indicators such as anti-tensile strength and internal bonding strength of the paper were measured. The anti-tensile strength was evaluated after setting the anti-tensile strength parameters (interval: 10 mm), cutting paper into a sample with a length of 170 mm and a width of 15 mm, and fixing the same. The sample size used in measuring the internal bursting strength was measured at 100 mm×100 mm. The internal bursting strength index was calculated as follows.

Internal bursting strength index X=P/W (X: Internal bursting strength index kPa·m²/g; P: Internal bursting strength kPa; W: Paper weight, g/m²)

In addition, the interlayer bonding strength sample was sheared to a size of 25.4 mm×140 mm, and measured using an internal bond strength meter. The results are shown in Tables 7 and 8 below.

TABLE 7

| Product | T-C % | Activity |
|---|---|---|
| Concentrate | 0.23 | 473,443 |
| Decolorizing | 1.62 | 479,384 |
| solution | | |
| Concentrate | 1.08 | 118,156 |
| (diluted) | | |
| Decolorizing | 33.61 | 115,989 |
| solution (diluted) | | |

TABLE 8

| | | | | | | Internal |
|---|---|---|---|---|---|---|
| | | | | | | bursting |
| | | | | | Internal | strength |
| | | Disso- | Gram | Tensile | bond | index |
| Enzyme | Pulping | ciation/ | weight | strength/ | strength | kPa |
| (diluted) | PRM/r | ml | g/m² | km | J/m² | m²/g |
| Before | 7000 | 330 | 89.17 | 4.646 | 146.8 | 3.234 |
| decolorizing | | | | | | |
| After | 7000 | 336 | 87.89 | 4.613 | 142.7 | 3.167 |
| decolorizing | | | | | | |

As shown in Tables 7 and 8, it was confirmed that there was no change in the enzyme performance before and after decolorizing.

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds are therefore intended to be embraced by the claims.

### Effect of the Invention

A method of decolorizing a composition including pectate lyase of the present disclosure employs dicyandiamide and hydrogen peroxide, thereby effectively removing dyes generated as fermentation by-products in the decolorizing process during the production of enzymes while solving the problem of enzyme deactivation during the decolorizing process, and therefore, it may be usefully applied to the industrial production of pectate lyase.

What is claimed is:

1. A method of decolorizing a composition comprising pectate lyase, the method comprising the steps of:
preparing the composition comprising pectate lyase by culturing a microorganisms of the genus Bacillus in a medium; and
decolorizing the composition comprising pectate lyase by adding dicyandiamide and hydrogen peroxide thereto.

2. The method of claim 1, wherein the composition comprising pectate lyase comprises the microorganism of the genus Bacillus, a culture of the microorganism, a fermentation product of the microorganism, or a combination thereof.

3. The method of claim 1, wherein the dicyandiamide is comprised in an amount of 0.1 part by weight to 5 parts by weight, based on the total 100 parts by weight of the composition.

4. The method of claim 1, wherein the dicyandiamide is comprised in an amount of 0.1 part by weight to 5 parts by weight, based on the total 100 parts by weight of the composition, and
the hydrogen peroxide is comprised in an amount of 0.1 M to 1 M.

5. The method of claim 1, wherein the composition comprising dicyandiamide and hydrogen peroxide has increased stability of pectate lyase after the decolorizing step, as compared to a composition not comprising dicyandiamide and hydrogen peroxide.

6. The method of claim 1, wherein the composition comprising pectate lyase after the decolorizing step maintains 90% or more of the activity of pectate lyase, as compared to the composition before the decolorizing step.

7. A composition for decolorizing a composition comprising pectate lyase, the composition comprising dicyandiamide and hydrogen peroxide.

8. The composition of claim 7, wherein the pectate lyase is derived from a microorganism of the genus Bacillus.

9. The composition of claim 7, wherein the composition comprising pectate lyase comprises the microorganism of the genus Bacillus, a culture of the microorganism, a fermentation product of the microorganism, or a combination thereof.

10. The composition of claim 7, wherein the dicyandiamide is comprised in an amount of 0.1 part by weight to 5 parts by weight, based on the total 100 parts by weight of the composition.

11. The composition of claim 7, wherein the dicyandiamide is comprised in an amount of 0.1 part by weight to 5 parts by weight, based on the total 100 parts by weight of the composition, and
the hydrogen peroxide is comprised in an amount of 0.1 M to 1 M.

12. The composition of claim 7, wherein the composition has improved stability of pectate lyase, as compared to a composition not comprising dicyandiamide and hydrogen peroxide.
